(19) United States
(12) Patent Application Publication
(10) Pub. No.: US 2025/0042828 A1
(43) Pub. Date: Feb. 6, 2025

(54) METHOD FOR TREATING A GASEOUS COMPOSITION COMPRISING PROPANE

(71) Applicant: Neste Oyj, Espoo (FI)

(72) Inventors: Ville SUNTIO, Porvoo (FI); Victor SIFONTES HERRERA, Porvoo (FI)

(73) Assignee: Neste Oyj, Espoo (FI)

(21) Appl. No.: 18/719,090

(22) PCT Filed: Dec. 2, 2022
(86) PCT No.: PCT/FI2022/050806

§ 371 Date: Jun. 12, 2024

(30) Foreign Application Priority Data
Dec. 17, 2021 (FI) 20216296

(51) Int. Cl. C07C 7/04; C07C 7/00; C07C 7/09

(52) U.S. Cl. CPC C07C 7/04; C07C 7/005; C07C 7/09

(57) ABSTRACT

A method for treating a gaseous composition is herein disclosed. The method includes subjecting a gaseous composition comprising H2, methane, ethane, propane, and hydrocarbons having a carbon number of at least C4 to distillation in a thermally coupled distillation system to recover a propane composition.

### TECHNICAL FIELD

The present disclosure generally relates to a method for treating a gaseous composition and to a method for co-producing a propane composition and a fuel component. The disclosure relates particularly, though not exclusively, to a method for treating a gaseous composition derived from a hydrotreatment effluent.

### BACKGROUND

This section illustrates useful background information without admission of any technique described herein representative of the state of the art.

Propane is typically used for example for residential heating or as a transportation fuel.

Traditionally, propane has been produced from crude mineral oil with well-established techniques and processes. However, efforts have been made to replace propane derived from mineral or fossil oil with more environmentally sustainable propane at least partially derived from renewable materials of biological origin.

The established processes for obtaining propane from crude mineral oil cannot easily be transferred to renewable materials, for example as renewable materials usually contain significant amounts of oxygen-containing organic compounds and have a carbon number distribution which significantly differs from the carbon number distribution of crude mineral oil.

There thus remains a need for methods for obtaining propane compositions especially from renewable materials.

### SUMMARY

It is an object to provide an improved method for separation of a propane composition from a gaseous composition. It is an object to reduce energy consumption of such method. It is an object to provide an improved, commercial scale process, especially for co-producing fuel components, such as aviation, gasoline and/or diesel fuel components, and high quality propane composition, usable for example for catalytic upgrading, such as for catalytic dehydrogenation to produce propene.

The appended claims define the scope of protection. Any examples and technical descriptions of apparatuses, products and/or methods in the description and/or drawings not covered by the claims are presented as examples useful for understanding the invention.

According to a first example aspect there is provided a method for treating a gaseous composition, the method comprising:
(i) providing a gaseous composition comprising H₂, methane, ethane, propane, and hydrocarbons having a carbon number of at least C4, wherein the total amount of H₂, methane, ethane, propane, and hydrocarbons having a carbon number of at least C4 is at least 80 wt-%, preferably at least 85 wt-%, more preferably at least 90 wt-% of the total weight of the gaseous composition; and
(ii) subjecting the gaseous composition to distillation in a thermally coupled distillation system comprising n distillation columns and at least one and at most n−1 condenser(s) and at least one and at most n−1 reboiler(s), n being an integer ≥2, to recover a propane composition.

According to a second example aspect there is provided a method for co-producing a propane composition and a fuel component comprising:
(i) subjecting a hydrotreatment feed comprising vegetable oils, animal fats and/or microbial oils, optionally with a hydrocarbon diluent, to a catalytic hydrotreatment comprising at least hydrodeoxygenation using a sulphided hydrotreatment catalyst, to obtain a hydrotreatment effluent;
subjecting the hydrotreatment effluent to gas-liquid separation to obtain at least a gaseous stream comprising H₂, H₂S, CO, CO₂, H₂O, methane, ethane, propane and hydrocarbons having a carbon number of at least C4, and a liquid hydrotreated stream comprising C6-C30 hydrocarbons;
subjecting the gaseous stream to a pretreatment comprising at least purification to remove H₂S and CO₂, H₂ separation and drying to obtain a gaseous composition comprising H₂, methane, ethane, propane, and hydrocarbons having a carbon number of at least C4, wherein the total amount of H₂, methane, ethane, propane, and hydrocarbons having a carbon number of at least C4 is at least 80 wt-%, preferably at least 85 wt-%, more preferably at least 90 wt-% of the total weight of the gaseous composition; and
(ii) subjecting the gaseous composition to distillation in a thermally coupled distillation system comprising at least a first distillation column connected to a condenser without being connected to a reboiler, and a second distillation column connected to a reboiler without being connected to a condenser, by feeding the gaseous composition to the first distillation column, and recovering a propane composition from the second distillation column; and
wherein the method further comprises subjecting the liquid hydrotreated stream comprising C6-C30 hydrocarbons, optionally after a further catalytic hydrotreatment comprising at least hydroisomerisation, to a fractionation to recover one or more of a gasoline fuel component, an aviation fuel component, and/or a diesel fuel component.

Different non-binding example aspects and embodiments have been illustrated in the foregoing. The embodiments in the foregoing are used merely to explain selected aspects or steps that may be utilized in different implementations. The embodiments and preferred embodiments disclosed in relation to the present method for treating a gaseous composition, apply equally to the present method for co-producing a propane composition and a fuel component.

### BRIEF DESCRIPTION OF THE FIGURES

Some example embodiments will be described with reference to the accompanying figures, in which:

FIG. 1 shows a schematic drawing of the thermally coupled distillation of step (ii) according to an example embodiment;

FIG. 2 shows a schematic drawing of an example embodiment of the method of the present disclosure.

### DETAILED DESCRIPTION

In the following description, like reference signs denote like elements or steps.

All standards referred to in this text are the latest versions available at the filing date. C4+ compounds refer in the context of this disclosure to compounds having a carbon number of at least C4. C4+ hydrocarbons refer in the context of this disclosure to hydrocarbons having a carbon number of at least C4. C4+ hydrocarbon content refers to the wt-% content of C4+ hydrocarbons in the gaseous composition to be treated, any other streams within the present process or in the produced propane composition relative to the total weight of stream in question. Said C4+ hydrocarbon content is determined as sum amount of individual contents of components for each carbon number, such as (C4+C5+ . . . ) and so on. C5+ hydrocarbon content is used respectively for hydrocarbons having a carbon number of at least C5.

C5+ compounds refer in the context of this disclosure to compounds having a carbon number of at least C5. C5+ hydrocarbons refer in the context of this disclosure to hydrocarbons having a carbon number of at least C5.

A thermally coupled distillation system refers in the context of this disclosure to a distillation system that comprises n distillation columns and at least one and at most n−1 condenser(s) and at least one and most n−1 reboiler(s), n being an integer ≥2. In other words, in a thermally coupled distillation system, at least two columns share a condenser and a reboiler so that for said (at least) two distillation columns, there is just one condenser and one reboiler. In the experimental part, a system comprising two distillation columns, one condenser and one reboiler is studied as an example of the present thermally coupled distillation system.

Condenser column refers in the context of this disclosure to a distillation column (directly) connected, preferably at its top section, to a condenser and without being (directly) connected to a reboiler. Reboiler column refers in the context of this disclosure to a distillation column (directly) connected, preferably at its bottom section, to a reboiler and without being (directly) connected a condenser. Thus, there is no reboiler for example at the bottom section of the condenser column, and there is no condenser for example at the top section of the reboiler column.

Bottom section of a distillation column refers in the context of this disclosure to the section below the inlet of the gaseous composition and below the outlet of the propane composition, in the respective distillation column. Top section of a distillation column refers in the context of this disclosure to the section above the inlet of the gaseous composition and above the outlet of the propane composition, in the respective distillation column.

A conventional distillation system refers in the context of this disclosure to a distillation system comprising n distillation columns, n reboilers and n condensers, n being an integer number ≥1. In other words, a conventional distillation system comprises as many reboilers and as many condensers as there are distillation columns in the system.

Propane recovery is in the context of this disclosure determined by dividing the weight of propane in the recovered propane composition with the weight of propane in the gaseous composition subjected to the distillation of step (ii). The quotient may optionally be multiplied with 100% to express the propane recovery as wt-%.

As used in the context of this disclosure, unless specified otherwise, a “content ratio” refers to the ratio on a weight basis of contents of the specified components (wt-%/wt-%), and, unless otherwise specified, a “content” is a content on a weight basis and is calculated relative to the total weight of the composition in question, such as the total weight of the gaseous composition or the propane composition.

As used in the context of this disclosure, aviation fuel component refers to hydrocarbon components suitable for use in fuel compositions meeting standard specifications for aviation fuels, such as specifications laid down in ASTM D7566-2021. Typically, such aviation fuel components boil within the range from about 100° C. to about 300° C., such as within the range from about 150° C. to about 300° C., as measured according to EN ISO 3405-2019.

As used in the context of this disclosure, diesel fuel component refers to hydrocarbon compositions suitable for use in fuel compositions meeting standard specifications for diesel fuels, such as specifications laid down in EN 590-2013+A1-2017. Typically such diesel fuel components boil within the range from about 160° C. to about 380° C., as measured according to EN ISO 3405-2019.

As used in the context of this disclosure, gasoline fuel component refers to hydrocarbon compositions suitable for use in fuel compositions meeting standard specifications for gasoline fuels, such as specifications laid down in EN 228-2012+A1-2017. Typically, such gasoline fuel components boil within the range from about 25° C. to about 200° C., as measured according to EN ISO 3405-2019. Boiling temperatures of the fuels and fuel components refer to temperatures under normal atmospheric pressures, for example as measured according to EN ISO 3405-2019, unless otherwise provided.

The term renewable or bio-based or biogenic indicates presence of compounds or components derived from renewable sources (biological sources). Carbon atoms of renewable or biological origin (biogenic carbon) comprise a higher number of unstable radiocarbon (¹⁴C) atoms compared to carbon atoms of fossil origin. Therefore, it is possible to distinguish between carbon compounds derived from renewable or biological raw material and carbon compounds derived from fossil raw material by analysing the ratio of ¹²C and ¹⁴C isotopes. Thus, a particular ratio of said isotopes can be used as a “tag” to identify renewable carbon compounds and differentiate them from non-renewable carbon compounds. The isotope ratio does not change in the course of chemical reactions. Examples of a suitable method for analysing the content of carbon from biological or renewable sources are DIN 51637 (2014), ASTM D6866 (2020) and EN 16640 (2017). In the context of the present disclosure, the content of carbon from biological or renewable raw material (biological origin) is expressed as the biogenic carbon content meaning the amount of biogenic carbon in the material as a weight percent of the total carbon (TC) in the material as determined according to EN 16640 (2017). A biogenic carbon content of the total carbon content in a product, which is completely of biological origin, may be about 100 wt-%. The biogenic carbon contents of renewable hydrotreatment feed, diluent, gaseous composition, propane composition, and/or fuel components according to the present disclosure are lower in cases where other carbonaceous components besides biological or renewable components are used in the methods and/or in the propane composition of the present disclosure, but the biogenic carbon contents are preferably at least 5 wt-%.

There is provided a method for treating a gaseous composition, comprising:
(i) providing a gaseous composition comprising H₂, methane, ethane, propane, and hydrocarbons having a carbon number of at least C4, wherein the total amount of H₂, methane, ethane, propane, and hydrocarbons having a carbon number of at least C4 is at least 80 wt-%, preferably at least 85 wt-%, more preferably at least 90 wt-% of the total weight of the gaseous composition; and
(ii) subjecting the gaseous composition to distillation in a thermally coupled distillation system comprising n distillation columns and at least one and at most n−1 condenser(s) and at least one and at most n−1 reboiler(s), n being an integer ≥2, to recover a propane composition.

The total amount of H₂, methane, ethane, propane, and hydrocarbons having a carbon number of at least C4 is determined as a sum amount of said individual component weights in relation to the total weight of the gaseous composition.

The present disclosure provides a method for obtaining from the gaseous composition a propane composition with a high degree of purity, such as at least 95 wt-% propane based on the total weight of the propane composition, while maintaining high propane recovery, even 95 wt-% or more. In particular, the present disclosure provides a method for obtaining a high purity propane composition even from gaseous composition comprising elevated amounts, even about 25 wt-%, of C4+ hydrocarbons. The higher the amount of C4+ hydrocarbons, and especially the amount of C5+ hydrocarbons, in the gaseous composition, the more challenging it will be to achieve high purity propane composition meeting specifications for high-end uses, such as for catalytic upgrading, e.g. dehydrogenation of propane. Prior art processes have had difficulties reaching purity specifications without sacrificing propane recovery and yields of the propane composition. Subjecting the gaseous composition to distillation in a thermally coupled distillation system provides good control of product quality, i.e. quality of the recovered propane composition, while maintaining a high yield of the propane composition and high propane recovery. Also, cooling and reboiling duties are reduced compared to distillation in a conventional distillation system, such as distillation in a single cryogenic distillation. For example, the recovered propane composition may even without further purification fulfil one or more of EN 589, DIN 51622, BS 4250 or FID-5 propane specifications. On-specification propane compositions may thus be obtained with improved propane recovery or yield of the propane composition, and with lower energy consumption, i.e. with reduced condenser and reboiler duties, compared to distillation in for example a single conventional cryogenic distillation column. Furthermore, the present method provides enhanced control, so that for example each of the propane content, C4+ hydrocarbon content and C5+ hydrocarbon content in the propane composition can be provided on-specification or as targeted, without having to exceed the specification or target for any of these. The method of the present disclosure is particularly advantageous for separating a high purity propane composition from gaseous compositions comprising relatively high amounts of C4+ hydrocarbons. Such gaseous compositions may be, but are not limited to, gaseous streams separated from hydrotreatment effluents obtained during manufacturing of renewable fuel components.

The present method using the thermally coupled distillation system is thus suitable for producing high quality propane compositions in a cost-efficient manner, including meeting even stringent propane specifications for high-end uses, yet avoiding over-purification that would ultimately reduce the yield of the propane composition without any added value to the propane composition. Prior art methods using conventional distillation, such as distillation in a single cryogenic distillation column, have typically allowed optimization of the level of only one impurity, i.e. the most critical impurity level for meeting the targeted product specifications, sometimes referred to as limiting specification. This has resulted in producing over-quality regarding other impurities, or in quality trade-off by giving in regarding the limiting specification, potentially still over-purifying in other aspects (but to lesser extent). The prior art methods have thus often led to quality trade-off and/or reduced yield of the propane composition.

The propane composition recovered in the present method may comprise, based on the total weight of the propane composition, at least 95 wt-%, preferably at least 96 wt-% propane, and at most 5.0 wt-%, preferably at most 3.0 wt-% hydrocarbons having a carbon number of at least C4, and at most 0.20 wt-%, preferably at most 0.18 wt-%, more preferably at most 0.13 wt-% hydrocarbons having a carbon number of at least C5.

The gaseous composition may have a biogenic carbon content of at least 50 wt-%, preferably at least 75 wt-%, more preferably at least 90 wt-%, even more preferably at least 95 wt-%, based on the total weight of carbon (TC) in the gaseous composition. The propane composition recovered in the present method may have a biogenic carbon content of at least 50 wt-%, preferably at least 75 wt-%, more preferably at least 90 wt-%, even more preferably at least 95 wt-%, based on the total weight of carbon (TC) in the propane composition. In such embodiments, the gaseous composition or the propane composition may be fully renewable (having biogenic carbon content of e.g. about 100 wt-%) or a blend of renewable and fossil material.

In certain embodiments, the propane composition recovered in the present method is a propane composition meeting one or more of EN 589, DIN 51622, BS 4250 or FID-5 propane specifications (without further purification).

The content of methane, ethane, propane, hydrocarbons having a carbon number of at least C4, hydrocarbons having a carbon number of at least C5, and/or of unsaturated hydrocarbons (if present) in the propane composition may be determined in accordance with ASTM D 2163. The content of CO₂ in the propane composition may be determined in accordance with ASTM D 2505. The content of CO in the propane composition may be determined in accordance with ASTM D 2504. The content of sulphur containing compounds in the propane composition (such as H₂S or COS), calculated as elemental S, may be determined in accordance with ASTM D 6667. The content of water in the propane composition may be determined in accordance with ASTM D 5454.

The propane composition recovered in the present method may optionally be compressed, during or after the distillation of step (ii), in order to provide a liquefied propane composition. The compressing may be conducted after the distillation or may be conducted in the course of the distillation, e.g. in case the propane composition is in liquid state under the conditions employed in the distillation at least at the stage where propane composition is withdrawn.

The recovered propane composition may be formulated into a propane containing product. The propane containing product may comprise an odorizing agent, for example selected from one or more of: tert-butylthiol, tetrahydrothiophene and ethanethiol. The propane composition or propane containing product may be usable for example for heating, in a fuel for a vehicle, or for cooking.

The propane composition, in gas or liquid from, may be subjected to further processing. For example, it may be subjected to a conversion, such as hydroreforming, comprising catalytic dehydrogenation to obtain a dehydrogenated product/effluent, from which effluent at least a fraction comprising propene is recovered to obtain, after optional purification and/or fractionation, a propene composition. Propene composition may be used in polymerisation reactions or to form polymers, with or without co-monomers.

In certain preferred embodiments, the thermally coupled distillation system employed in step (ii) comprises at least a first distillation column (directly) connected, preferably at its top section, to a condenser without being (directly) connected to a reboiler, and a second distillation column (directly) connected, preferably at its bottom section, to a reboiler without being (directly) connected to a condenser. Hence, according to these preferred embodiments, the first distillation column is a condenser column, and the second distillation column is a reboiler column as defined in the present disclosure. The condenser column may thus be provided without a reboiler at its bottom section, and the reboiler column may thus be provided without a condenser at its top section. Consequently, vapor flow(s) between the condenser column and the reboiler column are unidirectional from the reboiler column to the condenser column, and liquid flow(s) between the condenser column and the reboiler column are unidirectional from the condenser column to the reboiler column. Unidirectional flow(s) are easier to implement and allow for an easier distillation setup than bidirectional flows. In these preferred embodiments, the gaseous composition is fed to the first distillation column, and the propane composition is recovered from the second distillation column. In certain other embodiments the gaseous composition may be fed to a reboiler column as the first distillation column, and the propane composition may be recovered from a condenser column as the second distillation column.

The thermally coupled distillation system of step (ii) may comprise one or more further distillation columns (in addition to the condenser column and the reboiler column). Such further distillation column(s) may or may not be thermally coupled (with each other or with the condenser column or with the reboiler column).

In certain embodiments, a liquid feed and a liquid reflux are provided from the condenser column to the reboiler column, and a vapour feed and a vapour boilup are provided from the reboiler column to the condenser column. In preferred embodiments, condenser column bottom is provided as the liquid feed to the bottom section of the reboiler column, and reboiler column overhead is provided as the vapour feed to the top section of the condenser column. Preferably the liquid reflux is provided from the top section of the condenser column, below the inlet of the vapour feed, to the top section of the reboiler column, and the vapour boilup is provided from the bottom section of the reboiler column, above the inlet of the liquid feed, to the bottom section of the condenser column. Any one or any combination of the liquid feed or liquid reflux or the vapour feed or the vapour boilup may be referred to as connecting stream(s) between the columns.

The reboiler column, the condenser column, and/or the optional further distillation column(s) may be, independently of each other, packed column(s), or tray or plate column(s), or any other suitable distillation column(s). Preferably, the reboiler column, the condenser column, and/or the optional further distillation column(s) are suitable for elevated pressure distillation (distillation above-atmospheric pressures). In certain embodiments, the condenser column and the reboiler column are both provided without a dividing wall or walls, i.e. they do not comprise a dividing wall or dividing walls. Also, the optional further distillation column(s) may be provided without a dividing wall or walls.

In certain embodiments, the condenser column is configured to separate from propane compounds lighter than propane, such as ethane, methane, and H₂, and the reboiler column is configured to separate from propane compounds heavier than propane, such as hydrocarbons having a carbon number of at least C4. Thus, the condenser column may form a rectifying section of the thermally coupled distillation system, and the reboiler may form a stripping section of the thermally coupled distillation system.

The liquid reflux may be provided from the condenser column to the reboiler column as one or more liquid reflux streams using one or more conduits, respectively, and similarly the vapour boilup may be provided from the reboiler column to the condenser column as one or more vapour boilup streams. Preferably, the thermally coupled distillation system is arranged so that one liquid reflux stream is provided from the condenser column to the reboiler column, and/or one vapour boilup stream is provided from the reboiler column to the condenser column. In this way the complexity of the columns is minimized and controlling is simpler.

The condenser column bottom, or the liquid feed to the reboiler column, may comprise mostly propane, such as 80 wt-% propane, together with C4+ hydrocarbons, and at most traces of compounds lighter than propane, such as CO, CO₂, CH₄, ethane, and/or H₂. The vapour boilup to the condenser column may comprise mostly propane, and small amounts of C4+ hydrocarbons, and of compounds lighter than propane, such as CO, CO₂, CH₄, ethane, and/or H₂. Also the liquid reflux to the reboiler column may comprise propane and C4+ hydrocarbons. The reboiler column overhead, or the vapour feed to the condenser column, may comprise propane, and small amounts of components lighter than propane, such as ethane, methane, CO, CO₂, and H₂, and small amounts of C4+ hydrocarbons.

The composition of the liquid stream(s), particularly the liquid feed and the liquid reflux, as well as of the vapour stream(s), particularly the vapour feed and the vapour boilup, between the condenser column and reboiler column may vary or fluctuate depending e.g. on process conditions, such as flow rate, pressures and temperatures and/or on the composition of the gaseous composition subjected to the distillation in step (ii).

The liquid stream(s) may be conveyed between the condenser column and the reboiler column by pump(s), or by natural convection facilitated e.g. by gravity. The vapour stream(s) between the condenser column and the reboiler column may be conveyed by natural convection facilitated by pressure difference, or for example by using one or more compressors. In preferred embodiments, vapour stream(s) between the condenser column and the reboiler column are arranged to be conveyed by pressure difference between the condenser column and the reboiler column.

Typically, the pressures in the thermally coupled distillation system are controlled with an overall pressure controller, and are influenced for example by the pressure drops caused by the column internals, valves etc. Temperatures in the thermally coupled distillation system are influenced for example by the pressures, and by the composition of the stream(s). The temperature and/or pressure conditions in the thermally coupled distillation system may be adjusted also by conditioning the connecting stream(s) between the columns, for example with compressor(s) and/or heat exchanger(s). By controlling e.g. the flow rates of the stream(s), it is possible to influence the separation and the temperatures.

The distillation in step (ii) may be conducted ensuring sufficient theoretical plates in the condenser column and the reboiler column so that compounds lighter than propane, such as ethane, methane, CO, CO₂, and H₂, and compounds heavier than propane, such as C4+ hydrocarbons, can be separated from propane.

In certain preferred embodiments, the distillation in step (ii) may be performed under above-atmospheric pressures (pressures above 1 atm). The condenser column and the reboiler column may be pressurised distillation columns. Typically, there is a vertical pressure gradient within the reboiler column, particularly over the combination of the reboiler column and the reboiler, and/or a vertical pressure gradient within the condenser column, particularly over the combination of the condenser column and the condenser. The pressure gradient may be influenced e.g. by the number of stages or column length.

In certain embodiments, the thermally coupled distillation system in step (ii) is operated at pressures above 1500 kPa, preferably above 1900 kPa, such as at pressures within a range from 1500 kPa to 5000 kPa, or from 1900 kPa to 4500 kPa, or from 2400 kPa to 3900 kPa.

In certain embodiments, the thermally coupled distillation system in step (ii) is operated at temperatures above −70° C., such as above −60° C., preferably within the range from −70° C. to 250° C., more preferably from −70° C. to 200° C., even more preferably from −70° C. to 180° C. If the temperature is much lower than −70° C., for example CO may start to solidify causing problems in the thermally coupled distillation system. The combination of the condenser column and the condenser may be operated at temperatures within a range from −70° C. to 120° C., preferably from −70° C. to 100° C. while the combination of the reboiler column and the reboiler may be operated at temperatures within a range from 0° C. to 250° C., preferably from 0° C. to 200° C., more preferably from 0° C. to 180° C.

In certain preferred embodiments, the thermally coupled distillation system in step (ii) is operated at pressures above 1500 kPa, preferably above 1900 kPa, such as at pressures within a range from 1500 kPa to 5000 kPa, or from 1900 kPa to 4500 kPa, or from 2400 kPa to 3900 kPa, and at temperatures above −70° C., such as above −60° C., preferably within the range from −70° C. to 250° C., more preferably from −70° C. to 200° C., even more preferably from −70° C. to 180° C.

In certain preferred embodiments, the combination of the condenser column and the condenser is operated at pressures within a range from 1500 kPa to 5000 kPa, or from 1900 kPa to 4500 kPa, or from 2400 kPa to 3900 kPa, and at temperatures within the range from −70° C. to 120° C., preferably from −70° C. to 100° C. Preferably the lowest temperature in the combination of the condenser column and the condenser is below 0° C. By operating the combination of the condenser column and the condenser at cryogenic conditions, it is possible to improve the propane recovery and the yield of the propane composition.

In certain embodiments, the thermally coupled distillation system in step (ii) is operated so that the lowest pressure in the combination of the reboiler column and the reboiler is higher than the highest pressure in the combination of the condenser column and the condenser, the lowest pressure in the combination of the reboiler column and the reboiler preferably being from 20 kPa to 150 kPa higher, more preferably from 30 kPa to 120 kPa higher, than the highest pressure in the combination of the condenser column and the condenser. When pressure in the combination of the reboiler column and the reboiler is higher than pressure in the combination of the condenser column and the condenser, no compressor(s) are necessarily needed, but the vapour stream(s) may be conveyed from the reboiler column to the condenser column by natural convection facilitated by the pressure difference, thereby reducing energy consumption. A moderate pressure difference, such as less than 120 kPa or within the range from 30 kPa to 120 kPa, is preferred because a much higher pressure difference between the combination of the reboiler column and the reboiler, and the combination of the condenser column and the condenser, could increase energy consumption and could require an increase of distillation temperature(s), which might complicate the setup of the thermally coupled distillation system. Nevertheless, it is also possible to operate the thermally coupled distillation system so that the lowest pressure in the combination of the reboiler column and the reboiler is not higher than the highest pressure in the combination of the condenser column and the condenser, and to convey the vapour stream(s) from the reboiler column to the condenser column e.g. by using one or more compressors.

Both the combination of the condenser column and the condenser, and the combination of the reboiler column and the reboiler, may have relatively high/wide temperature gradients. Typically, the temperature profile is steeper closer to the condenser stage or the reboiler stage, while otherwise the temperature profile may be less steep, especially when mainly separating C3 from C4 and C2 having boiling points relatively close to each other.

In certain embodiments, the thermally coupled distillation system is operated using a greater temperature gradient (Tmax−Tmin), and preferably also a greater pressure gradient (pmax−pmin), in the combination of the condenser column and the condenser than in the combination of the reboiler column and the reboiler.

In certain embodiments, the combination of the reboiler column and the reboiler is operated at pressures within a range from 1500 kPa to 5000 kPa, or from 1900 kPa to 4500 kPa, or from 2400 kPa to 3900 kPa, and at temperatures within a range from 0° C. to 250° C., preferably from 0° C. to 200° C., more preferably from 0° C. to 180° C. Preferably the lowest temperature in the combination of the condenser column and the condenser is below 0° C. and the highest temperature in the combination of the reboiler column and the reboiler is higher, preferably from 30° C. to 150° C., more preferably from 50° C. to 120° C. higher, than the highest temperature in the combination of the condenser column and the condenser, preferably the lowest temperature in the combination of the reboiler column and the reboiler being at most as high as the highest temperature in the combination of the condenser column and the condenser.

In addition to the propane composition also other compositions or streams may be recovered from step (ii). In certain embodiments, a stream of light compounds comprising e.g. H₂, CO, CO₂, CH₄, and ethane, may be recovered as the condenser column overhead vapour, and a stream of heavy compounds comprising C4+ hydrocarbons may be recovered as reboiler column bottom, from the thermally coupled distillation system of step (ii). The propane composition may be recovered from the reboiler column, e.g. from a product tray of the reboiler column.

The stream of light compounds exiting from the thermally coupled distillation system of step (ii) comprises typically CO, CO₂, CH₄, ethane, and H₂, and may also contain at least traces of propane. The stream of light compounds has a high heating value, and it may thus be burnt for energy. Optionally, as the stream of light compounds exiting from the thermally coupled distillation system of step (ii) may have a relatively high pressure, such as above 1500 kPa, preferably above 1900 kPa, such as within a range from 1500 kPa to 5000 kPa, or from 1900 kPa to 4500 kPa, or from 2400 kPa to 3900 kPa, the stream of light compounds may conveniently be subjected to H₂ separation e.g. by using a membrane separation technique.

The stream of heavy compounds exiting from the thermally coupled distillation system in step (ii) comprises C4+ hydrocarbons, and may also contain some propane. The stream of heavy compounds may for example be usable as a component in naphtha and/or in production of H₂ by steam reforming.

In certain embodiments, the gaseous composition provided in step (i) and subjected to distillation in step (ii) comprises propane at least 60 wt-%, preferably at least 65 wt-%, more preferably at least 70 wt-%, such as from 60 to 85 wt-%, or from 65 to 80 wt-%, or from 70 to 75 wt-%, based on the total weight of the gaseous composition. Propane being the main constituent of the gaseous composition enhances efficiency of the present method.

In certain embodiments, the gaseous composition provided in step (i) and subjected to distillation in step (ii) comprises hydrocarbons having a carbon number of at least C4 at most 18.5 wt-%, preferably at most 18.0 wt-%, more preferably at most 17.0 wt-%, even more preferably at most 16.0 wt-%, most preferably at most 10.5 wt-%, and/or at least 5.0 wt-%, preferably at least 7.0, more preferably at least 9.0 wt-%, such as from 5.0 to 18.5 wt-%, or from 7.0 to 17.0 wt-%, or from 9.0 to 16.0 wt-%, based on the total weight of the gaseous composition. The present method is especially beneficial in treating gaseous compositions having an elevated content of heavy tail (C4+), and is capable of achieving low targeted C4+ contents in the recovered propane composition.

In certain embodiments, the gaseous composition provided in step (i) and subjected to distillation in step (ii) has a content ratio of hydrocarbons having a carbon number of at least C4 to propane of at least 0.05, preferably within a range from 0.05 to 0.40, more preferably from 0.07 to 0.35, even more preferably from 0.09 to 0.30, most preferably from 0.10 to 0.20.

In certain embodiments, the gaseous composition provided in step (i) and subjected to distillation in step (ii) comprises hydrocarbons having a carbon number of at least C5 at most 15.0 wt-%, preferably at most 10.0 wt-%, more preferably at most 8.0 wt-%, even more preferably at most 6.0 wt-%, and/or at least 1.5 wt-%, preferably at least 2.0 wt-%, more preferably at least 3.0 wt-%, such as from 1.5 to 15.0 wt-%, or from 2.0 to 10.0 wt-%, or from 3.0 to 8.0 wt-%, based on the total weight of the gaseous composition. The present method is especially beneficial in treating gaseous compositions having an elevated content of heavy tail (C4+), particularly of C5+ hydrocarbons, and is capable of achieving very low targeted C5+ hydrocarbon contents in the recovered propane composition. Conventional distillation processes have difficulties to yield propane compositions with such a low content of especially C5+.

In certain embodiments, the gaseous composition provided in step (i) and subjected to distillation in step (ii) comprises hydrocarbons having a carbon number of at least C5, and has a content ratio of hydrocarbons having a carbon number of at least C5 to propane of at least 0.01, preferably within a range from 0.01 to 0.35, more preferably from 0.02 to 0.30, even more preferably from 0.03 to 0.25, most preferably from 0.04 to 0.15.

In certain embodiments, the gaseous composition provided in step (i) and subjected to distillation in step (ii) comprises compounds lighter than propane, such as H₂, methane and ethane, at most 35 wt-%, preferably from 5 to 30 wt-%, more preferably from 10 to 25 wt-%, based on the total weight of the gaseous composition, and/or H₂ from 1 to 10 wt-%, preferably from 2 to 9 wt-%, more preferably from 3 to 8 wt-%, based on the total weight of the gaseous composition. While the present method is capable of treating gaseous compositions having elevated content of compounds lighter than propane, at most moderate content of the compounds lighter than propane ensures lower condenser and reboiler duties, and allows using smaller equipment and higher pressures in the thermally coupled distillation system.

In certain embodiments, the gaseous composition provided in step (i) and subjected to distillation in step (ii) comprises CO at most 15 wt-%, preferably from 1 to 15 wt-%, more preferably from 2 to 10 wt-%, based on the total weight of the gaseous composition, and/or CO₂ from 0.01 to 5.0 wt-%, preferably from 0.1 to 3.0 wt-%, based on the total weight of the gaseous composition. CO and/or CO₂ are typical impurities in gaseous compositions obtainable by hydrotreating renewable hydrotreatment feeds such as vegetable oils, animal fats, microbial oils, etc as deoxygenation thereof generates not only H₂O but also varying amounts of CO and CO₂, depending on how much the hydrotreatment conditions favor decarbonylation/decarboxylation reactions. While sweetening e.g. using an amine scrubber may remove at least part of CO₂, some amounts thereof, and especially CO, typically remain in the gaseous composition. The present method is capable of removing both CO and CO₂ to the low levels as required by specifications for high-end uses of the propane compositions, such as for catalytic upgrading, e.g. dehydrogenation of propane to propylene.

In certain preferred embodiments, (i) providing the gaseous composition comprises subjecting a hydrotreatment effluent to gas-liquid separation to obtain at least a gaseous stream, and optionally subjecting the gaseous stream to a pretreatment to obtain the gaseous composition. In embodiments involving subjecting the gaseous stream to a pretreatment comprising at least H₂ separation, to obtain the gaseous composition, the separated H₂ or at least a portion thereof is preferably recycled to the hydrotreatment, so as to further improve the cost-efficiency and sustainability of the method. Furthermore, when H₂ is separated also from the stream of light compounds optionally recovered in step (ii), at least a portion of the H₂ separated from said stream of light compounds may be combined with at least a portion of the H₂ separated in the pretreatment in step (i), and preferably at least a portion of the combined H₂ stream is recycled to the hydrotreatment. Such recycling may help to capture some of the traces of C4+ hydrocarbons that may be present in the H₂ separated from the stream of light compounds in step (ii) and/or in the H₂ separated in the pretreatment in step (i).

Subjecting the gaseous stream to a pretreatment comprising at least H₂ separation is beneficial, as gaseous streams separated from hydrotreatment effluents may have high H₂ contents, while treating in step (ii) gaseous compositions having only moderate content of compounds lighter than propane provide the benefits as explained in the foregoing.

In certain embodiments, (i) providing the gaseous composition comprises subjecting a hydrotreatment feed comprising vegetable oils, animal fats, microbial oils, crude oil, thermally, such as thermocatalytically, and/or enzymatically liquefied organic waste and residues, such as biomass waste and residues, municipal solid waste and/or waste plastics, or a combination thereof, optionally with a hydrocarbon diluent, to a catalytic hydrotreatment comprising hydrodeoxygenation (HDO), hydrocracking, hydroisomerization, hydropyrolysis, hydrodesulphurization (HDS), hydrodenitrogenation (HDN), hydrodehalogenisation (HDX), hydrodearomatization (HDA), hydrodemetallation and/or hydrogenation, to obtain a hydrotreatment effluent; subjecting the hydrotreatment effluent to gas-liquid separation to obtain at least a gaseous stream; and subjecting the gaseous stream to a pretreatment to obtain the gaseous composition. Various sustainable and/or renewable materials may be used as the hydrotreatment feed, optionally with a hydrocarbon diluent especially for controlling temperature in exothermic hydrotreatments. Depending on the composition and/or impurities of the hydrotreatment feed, and on the other products that are desired to be produced, suitable catalytic hydrotreatment and conditions therefor may be selected. In certain preferred embodiments, (i) providing the gaseous composition comprises subjecting a hydrotreatment feed comprising vegetable oils, animal fats, microbial oils, and/or combinations thereof, optionally with a hydrocarbon diluent, to a catalytic hydrotreatment comprising at least hydrodeoxygenation, to obtain a hydrotreatment effluent; subjecting the hydrotreatment effluent to gas-liquid separation to obtain at least a gaseous stream and a liquid hydrotreated stream; subjecting the gaseous stream to a pretreatment to obtain the gaseous composition; and further subjecting at least a portion of the liquid hydrotreated stream to a further catalytic hydrotreatment comprising at least hydroisomerisation to obtain a further hydrotreatment effluent; subjecting the further hydrotreatment effluent to a further gas-liquid separation to obtain at least a further gaseous stream and a further liquid hydrotreated stream comprising isomerised C6-C30 hydrocarbons; combining at least a portion of the further gaseous stream with the gaseous stream before or after the pretreatment; and subjecting the further liquid hydrotreated stream comprising isomerized C6-C30 hydrocarbons to a fractionation to recover one or more of a gasoline fuel component, an aviation fuel component, and/or a diesel fuel component.

In certain preferred embodiments, (i) providing the gaseous composition comprises subjecting a hydrotreatment feed comprising vegetable oils, animal fats and/or microbial oils, optionally with a hydrocarbon diluent, to a catalytic hydrotreatment comprising at least hydrodeoxygenation using a sulphided hydrotreatment catalyst, to obtain a hydrotreatment effluent; subjecting the hydrotreatment effluent to gas-liquid separation to obtain at least a gaseous stream comprising H₂S, CO, CO₂, H₂O, methane, ethane, propane and hydrocarbons having a carbon number of at least C4, and a liquid hydrotreated stream comprising C6-C30 hydrocarbons; subjecting the gaseous stream to a pretreatment comprising at least purification to remove H₂S and CO₂, H₂ separation and drying to obtain dried H₂S, CO₂ and H₂ depleted gaseous stream as the gaseous composition; and wherein the method further comprises subjecting the liquid hydrotreated stream comprising C6-C30 hydrocarbons, optionally after a further catalytic hydrotreatment comprising at least hydroisomerisation, to a fractionation to recover one or more of a gasoline fuel component, an aviation fuel component, and/or a diesel fuel component.

The gaseous composition comprising H₂, methane, ethane, propane, and hydrocarbons having a carbon number of at least C4, wherein the total amount of H₂, methane, ethane, propane, and hydrocarbons having a carbon number of at least C4 is at least 80 wt-%, preferably at least 85 wt-%, more preferably at least 90 wt-% of the total weight of the gaseous composition, provided in step (i) may originate from hydrotreatment, particularly hydrotreatment of vegetable oils, animal fats and/or microbial oils. In particular, the gaseous composition provided in step (i) may be obtainable or obtained as a gaseous stream from gas-liquid separation of a hydrotreatment effluent, particularly from hydrotreatment of vegetable oils, animal fats and/or microbial oils. The hydrotreatment may be for example hydrotreatment to produce a fuel component, such as gasoline, diesel and/or aviation fuel component, preferably an aviation fuel component. For example, the gaseous composition could be a gaseous stream separated from a hydrotreatment effluent from a hydrotreatment as described in any one of F1100248, U.S. Pat. No. 8,859,832, U.S. Ser. No. 10/800,976, EP1741768, U.S. Ser. No. 10/941,349, U.S. Pat. No. 8,742,185, EP3517591, EP2141217, U.S. Pat. No. 5,705,722, CN107488462B, U.S. Pat. No. 9,567,264.

As relatively high amounts of C4+ hydrocarbons tend to end up in the gaseous stream of the hydrotreatment effluent when producing aviation range paraffins due to more stringent hydrotreatment conditions, the present method is particularly advantageous for recovering a high-purity or on specification propane composition from such gaseous fractions. With the present method, on specification propane composition may be recovered with a high propane recovery and yield of the propane composition and relatively low condenser and reboiler duties despite relatively high amounts of C4+ hydrocarbons in the gaseous fraction.

Hydrotreatment of a hydrotreatment feed, especially to produce fuel range hydrocarbons, particularly when the hydrotreatment feed comprises vegetable oils, animal fats, microbial oils, and/or combinations thereof, may involve generation of high amounts of gaseous reaction products ending up to the gaseous stream separated from the hydrotreatment effluent. Examples of these reaction products may include H₂O cleaved by hydrotreatment (HDO) from organic oxygenates such as fatty acids; CO and CO₂ cleaved by decarbonylation and decarboxylation of organic oxygenates such as fatty acids; propane, originating e.g. from glycerides and/or by cracking; various cracking products, including methane, ethane, C4+ hydrocarbons, of the organic oxygenates such as fatty acids or hydrocarbons obtained therefrom or used for diluting; H₂S cleaved by hydrodesulphurization from organic sulphur containing compounds present in some hydrotreatment feeds and/or sometimes added for maintaining activity of a sulphided hydrotreatment catalyst; and NH₃ cleaved by hydrodenitrogenation from organic nitrogen containing compounds typically present in renewable hydrotreatment feeds such as vegetable oils, animal fats, microbial oils etc. Additionally the gaseous stream separated from the hydrotreatment effluent may comprise significant amounts of unused (unreacted) hydrogen (H₂). The gaseous stream may contain hydrogen (H₂), based on the total weight of the gaseous stream, at least 70 mol-%, such as at least 75 mol-%, at least 80 mol-% and/or the H₂ content may be less than 95 mol-%, such as less than 90 mol-%.

Hydrotreating renewable hydrotreatment feeds such as vegetable oils, animal fats, microbial oils etc has gathered interest not just for producing renewable diesel fuel components but also renewable aviation fuel components. Hydrotreatment may be adjusted so that more cracking is achieved (compared to production of renewable diesel fuel components) in order to increase yields of typical aviation fuel range hydrocarbons having carbon chain lengths of C8-C15 from renewable hydrotreatment feeds such as vegetable oils, animal fats, microbial oils etc usually containing fatty acids with backbone carbon chain lengths of C16-C20. In addition to the desired aviation fuel range C8-C15 hydrocarbons also shorter hydrocarbons (hydrocarbons having a carbon number of at most C7) are formed due to the increased cracking achieved e.g. by using more severe hydrotreatment conditions, e.g. higher temperature and/or pressure, or catalysts or co-catalysts having higher cracking activity and/or selectivity.

Such methods are especially beneficial for co-producing a high quality propane composition and a fuel component from hydrotreatment feeds comprising vegetable oils, animal fats, microbial oils, and/or combinations thereof, as (compared to hydrotreatment optimized for producing diesel fuel range hydrocarbons) increased cracking during hydrotreatment to obtain more aviation fuel range paraffins may increase not only formation of C4+ hydrocarbons but also formation of propane (in addition to propane originating from glycerol-moieties of the feed), increasing the overall propane yield.

Furthermore, high quality propane composition may be produced even from gaseous compositions containing elevated amounts of entrained C4+ hydrocarbons, especially C4-C6 hydrocarbons, without a need to restrict recycling of gaseous and/or liquid streams in the method.

The present method is especially well suited for the varying conditions of real-life providing flexibility, so that the selection of the configuration of the units, process conditions, and/or variations in feed characteristics do not compromise the quality of the recovered propane composition.

Preferably a method is provided for co-producing a propane composition and a fuel component comprising:
(i) subjecting a hydrotreatment feed comprising vegetable oils, animal fats and/or microbial oils, optionally with a hydrocarbon diluent, to a catalytic hydrotreatment comprising at least hydrodeoxygenation using a sulphided hydrotreatment catalyst, to obtain a hydrotreatment effluent;
subjecting the hydrotreatment effluent to gas-liquid separation to obtain at least a gaseous stream comprising H₂, H₂S, CO, CO₂, H₂O, methane, ethane, propane and hydrocarbons having a carbon number of at least C4, and a liquid hydrotreated stream comprising C6-C30 hydrocarbons;
subjecting the gaseous stream to a pretreatment comprising at least purification to remove H₂S and CO₂, H₂ separation and drying to obtain a gaseous composition comprising H₂, methane, ethane, propane, and hydrocarbons having a carbon number of at least C4, wherein the total amount of H₂, methane, ethane, propane, and hydrocarbons having a carbon number of at least C4 is at least 80 wt-%, preferably at least 85 wt-%, more preferably at least 90 wt-% of the total weight of the gaseous composition; and
(ii) subjecting the gaseous composition to distillation in a thermally coupled distillation system comprising at least a first distillation column connected to a condenser without being connected to a reboiler, and a second distillation column connected to a reboiler without being connected to a condenser, by feeding the gaseous composition to the first distillation column, and recovering a propane composition from the second distillation column; and
wherein the method further comprises subjecting the liquid hydrotreated stream comprising C6-C30 hydrocarbons, optionally after a further catalytic hydrotreatment comprising at least hydroisomerisation, to a fractionation to recover one or more of a gasoline fuel component, an aviation fuel component, and/or a diesel fuel component.

In addition to recovering one or more fuel components, particularly in addition to recovering one or more of a gasoline fuel component, an aviation fuel component, and/or a diesel fuel component, including various grades of these fuel components, also other hydrocarbon compositions than fuel components and/or other fuel components than a gasoline fuel component, an aviation fuel component, or a diesel fuel component may be recovered from the fractionation of the methods according to the present disclosure, such as hydrocarbon compositions for electrotechnical fluids and/or a marine fuel component.

In certain embodiments, subjecting the gaseous stream to a pretreatment comprises at least purification to remove H₂S and CO₂, H₂ separation and drying to obtain dried H₂S, CO₂ and H₂ depleted gaseous stream as the gaseous composition. Any conventionally used pretreatment operations and equipment may be used to achieve this, such as those disclosed in WO2017045791 or WO2021110524. The pretreatment of the gaseous stream may comprise at least purification to remove H₂S and CO₂, and possibly also other impurities such as NH₃, for example by sweetening e.g. using an amine scrubber, or other conventional unit operations used in e.g. refineries. Sour gas, particularly the presence of H₂S, may be harmful for example to membrane material optionally employed in a subsequent H₂ separation which may be performed by membrane separation. Moreover, the presence of H₂S in addition to CO₂ may result in formation of COS which cannot be easily separated from propane by distillation. Since the formation of COS is an equilibrium reaction, it is shifted to the COS side by increasing the contents of CO₂ and H₂S and by decreasing the content of H₂O. Thus, by removing CO₂ and H₂S before drying, the formation of COS can be suppressed. The method of the present disclosure may comprise subjecting the H₂S and CO₂ depleted gaseous stream to H₂ separation and to drying to obtain a dried H₂S, CO₂ and H₂ depleted gaseous stream and a stream rich in H₂ as the separated H₂ stream.

The H₂ separation is preferably performed using a selective membrane (selective membrane separation). However, other methods for separating H₂ (and optionally at the same time other gaseous components) may be performed using any other suitable method, such as swing adsorption. The hydrogen selective membrane is preferably selective for hydrogen over propane, in that it preferentially permeates most of hydrogen and rejects most of propane and C4+ hydrocarbons in the retentate. The membrane is usually operated such that there will remain some hydrogen (H₂) in the retentate stream because it will result in a higher purity of hydrogen (H₂) in the permeate stream (stream rich in H₂). If present, CO and hydrocarbons lighter than propane may also be rejected together with propane, while H₂O, CO₂, H₂S and NH₃ may be rejected or partially rejected depending on the membrane type and conditions, e.g. temperature and pressure, of the membrane separation. A driving force for transmembrane permeation is provided by a higher pressure on the feed side than on the permeate side. For example, the pressure on the feed side may be 10 bar (gauge) or higher, such as 30 bar (gauge) or higher, or 50 bar (gauge) or higher, and the pressure on the permeate side may include a pressure that is at least 1 bar lower than a pressure on the feed side, such as at least 5 bar lower, or at least 10 bar lower, or at least 30 bar lower. The drying may be carried out before H₂ separation or the drying may be carried out after H₂ separation. In view of processing efficiency, it is preferred that the drying is carried out after H₂ separation. In this way smaller drying equipment is needed, requiring less space and lower investment cost. The drying may be carried out using any conventionally known chemical and/or physical method, e.g. using an adsorbent and/or absorbent for water. One particularly preferred embodiment involves drying using molecular sieve dehydration beds.

The hydrotreatment feed may comprise vegetable oils, animal fats, microbial oils, crude oil, thermally, such as thermocatalytically, and/or enzymatically liquefied organic waste and residues, such as biomass waste and residues, municipal solid waste and/or waste plastics, and/or any combinations thereof. Preferably a renewable hydrotreatment feed is used. Renewable hydrotreatment feed refers especially to a feedstock derived from biological raw material containing oil(s) and/or fat(s), usually containing free fatty acids and/or glycerides, such as plant oils/fats, vegetable oils/fats, animal oils/fats, fish oils/fats and/or algae oils/fats, and/or oils/fats from other microbial processes. Said oils/fats may include for example genetically manipulated algae oils/fats, genetically manipulated oils/fats from other microbial processes and/or also genetically manipulated vegetable oils/fats. Components of such materials could also be used, such as for example alkyl esters (typically C1 C5-alkyl esters, such as methyl, ethyl, propyl, iso-propyl, butyl, sec-butyl esters). Preferably a renewable hydrotreatment feed comprising vegetable oils, animal fats, microbial oils, and/or any combinations thereof, is used.

Examples of vegetable oils usable in the renewable hydrotreatment feed include, but are not limited to rapeseed oil, canola oil, soybean oil, coconut oil, sunflower oil, palm oil, palm kernel oil, peanut oil, linseed oil, sesame oil, maize oil, poppy seed oil, cottonseed oil, soy oil, tall oil, corn oil, castor oil, jatropha oil, jojoba oil, olive oil, flaxseed oil, camelina oil, safflower oil, babassu oil, Brassica carinata oil, and rice bran oil, and fractions and residues of above mentioned oils such as palm olein, palm stearin, palm fatty acid distillate (PFAD), purified tall oil, tall oil fatty acids, tall oil resin acids, tall oil unsaponifiables, tall oil pitch (TOP), and used cooking oil of vegetable origin. Examples of animal fats usable in the renewable hydrotreatment feed include, but are not limited to tallow, lard, yellow grease, brown grease, fish fat, poultry fat, and used cooking oil of animal origin. Examples of microbial oils usable in the renewable hydrotreatment feed include algal lipids, fungal lipids and bacterial lipids.

Vegetable oils, animal fats, microbial oils, and/or any combinations thereof typically comprise C10-C24 fatty acids, including esters of fatty acids, glycerides, i.e. glycerol esters of fatty acids, phospholipids, glycolipids, sphingolipids, etc. The glycerides may specifically include monoglycerides, diglycerides and triglycerides. Upon hydrogenation, the glycerol backbone of the glycerides is usually converted into renewable propane. Thus, the present disclosure also relates to a method for producing renewable propane composition from renewable hydrotreatment feeds.

The present disclosure provides a flexible process allowing easy adjustment of the composition of the hydrotreatment feed and operating conditions, thereby obtaining a gasoline fuel component, an aviation fuel component and/or diesel fuel component, and a high quality propane composition, in a ratio that best meets the prevailing or foreseen market demand.

The hydrotreatment feed or any of its constituent feeds, especially one or more of the feeds selected from vegetable oils, animal fats, microbial oils, crude oil, thermally, such as thermocatalytically, and/or enzymatically liquefied organic waste and residues, such as biomass waste and residues, municipal solid waste and/or waste plastics, and combinations thereof, may be subjected to a purifying pretreatment before subjecting to the hydrotreatment. Such purifying pretreatment may comprise one or more of washing, degumming, bleaching, evaporation, distillation, fractionation, rendering, heat treatment, filtering, adsorption, partial hydrodeoxygenation, partial hydrogenation, hydrolysis, transesterification, centrifugation and/or precipitation. These pre-treatment methods are simple and effective for removing impurities containing S, N, P, metals, and/or metalloids (such as Si), pitch, solids and/or compounds containing unsaturated bonds. Presence of these impurities in elevated amounts in the hydrotreatment feed may expedite deactivation of the hydrotreatment catalyst, and elevated content of compounds containing unsaturated bonds may complicate temperature control in the hydrotreatment.

The hydrotreatment feed or any of its constituent feeds may be combined with a hydrocarbon diluent before optional purification pretreatment. Alternatively or additionally the hydrotreatment feed may be combined with the diluent before the hydrotreatment or the diluent may be fed directly to the hdyrotreatment. The hydrocarbon diluent may be for example a diluent of mineral origin (fossil diluent), a diluent of biological origin (such as renewable paraffins) or preferably hydrocarbons separated and recycled from any of the product streams or effluents of the hydrotreatment. In case increased cracking occurs in the hydrotreatment, the recycled portions of the hydrotreatment effluent(s) may contain an increased amount of especially C4-C6 hydrocarbons. The present method is beneficial in that high purity propane composition may be produced with good yield and propane recovery despite such increase of C4-C6 hydrocarbons in the process feeds.

In the catalytic hydrotreatment a sulphided hydrotreatment catalyst may be employed. The sulphided state of the catalyst is preferably maintained by addition of a sulphur-containing compound to the hydrotreatment feed and/or to the hydrocarbon diluent and/or fed along the H₂ gas and/or separately to the hydrotreatment reactor. Typically, the sulphur containing compound is H₂S. In certain embodiments, the sulphur content of the hydrotreatment feed, calculated as elemental S, is from 10 to 10000 w-ppm, preferably from 10 to 1000 w-ppm, more preferably from 10 to 500 w-ppm, even more preferably from 10 to 300 w-ppm, yet more preferably from 10 to 200 w-ppm, and most preferably from 20 to 100 w-ppm. By adjusting the sulphur content within this range, the occurrence of decarb-reactions may be controlled or suppressed, and the lower sulphur content in the feed is beneficial also for controlling or suppressing generation of COS. That is, while a minimum amount of sulphur ensures sufficient catalyst activity in embodiments wherein a sulphided catalyst is employed in the hydrotreatment (without necessitating high temperatures), not exceeding a sulphur content of for example 10000 w-ppm or 1000 w-ppm may suppress the formation of (large amounts of) H₂S, which might convert into COS, so that less strenuous efforts are needed to reduce the amount of H₂S after hydrotreatment. The content of the sulphur in the hydrotreatment feed, calculated as elemental S, may be determined in accordance with EN ISO 20846. H₂S which may be removed when pretreating the gaseous stream in step (i) may be recovered and recycled into the hydrotreatment as a sulphur source for maintaining the activity of the sulphided metal catalyst employed therein.

It is preferred that the hydrotreatment conditions are selected such that the hydrotreatment provides saturated hydrocarbons (paraffins), especially n-paraffins and/or isoparaffins, preferably having carbon numbers in aviation fuel range.

Many conditions for hydrotreatment, such as hydrodeoxygenation or hydroisomerisation, are known to the skilled person. The hydrotreatment may be carried out in the presence of a catalyst, such as sulphided metal catalyst. The catalyst may comprise one or more Group VI metals, such as Mo or W, or one or more Group VIII non-noble metals such as Co or Ni. The catalyst may be supported on any convenient support, such as alumina, silica, zirconia, titania, amorphous carbon, molecular sieves or combinations thereof. Usually, the metal of the catalyst is impregnated or deposited on the support as metal oxide(s). In case sulphided metal catalyst is desired, the metal oxide(s) are then typically converted into their sulphides.

Examples of typical catalysts for hydrodeoxygenation are molybdenum containing catalysts, NiMo, CoMo, and/or NiW catalysts; supported on alumina or silica, but many other hydrodeoxygenation catalysts are known in the art and have been described together with or compared to NiMo and/or CoMo catalysts. The hydrodeoxygenation is preferably carried out under the influence of sulphided hydrotreatment catalysts such as catalysts comprising sulphided NiMo or sulphided CoMo, in the presence of hydrogen (H₂) gas. Examples of typical catalysts for hydroisomerisation, when performed, are catalyst containing SAPO-11 or SAPO-41 or ZSM-22 or ZSM-23 or ferrierite and Pt, Pd, or Ni and Al₂O₃ or SiO₂. The hydroisomerisation is preferably carried out under the influence of Pt/SAPO-11/Al₂O₃, Pt/ZSM-22/Al₂O₃, Pt/ZSM-23/Al₂O₃ or Pt/SAPO-11/SiO₂.

The hydrotreatment may be performed under a hydrogen pressure selected from a range from 10 to 200 bar, preferably from 30 to 100 bar, a temperature selected from a range from 200° C. to 500° C., preferably 250° C. to 400° C., and a feed rate (liquid hourly space velocity) of 0.1 to 10 h⁻¹ (v/v).

By feeding the hydrogen (H₂) to the hydrotreatment so as to provide a (H₂ partial) pressure selected from the range from 1 to 200 bar (or preferably from 10 to 100 bar, more preferably from 30 to 70 bar), efficient HDO, HDN (hydrodenitrification), and HDS (hydrodesulphurisation) reactions can be ensured while controlling decarb and/or cracking reactions keeping them at a low level.

In embodiments, wherein the hydrotreatment comprises hydrodeoxygenation and hydroisomerisation, the hydrodeoxygenation may be performed under a hydrogen pressure selected from a range from 10 to 100 bar, preferably from 30 to 70 bar, at a temperature selected from a range from 200° C. to 400° C., preferably from 250° C. to 350° C., more preferably from 280° C. to 340° C., and liquid hourly space velocities of 0.1 h⁻¹ to 10 h⁻¹, preferably 0.1 h⁻¹ to 3.0 h⁻¹, more preferably of 0.2 to 2.0 h⁻¹; and the hydroisomerisation may be performed under a hydrogen pressure selected from a range from 10 to 150 bar, preferably from 30 to 100 bar, at a temperature selected from a range from 200° C. to 500° C., preferably from 280° C. to 400° C., and liquid hourly space velocities of 0.1 h⁻¹ to 10 h⁻¹.

The present method provides flexibility as the severity of the hydrotreatment, e.g. of hydroisomerisation, may be smoothly adjusted depending on the market demand of an optionally recovered gasoline, aviation and/or diesel fuel component, without a need to essentially change or adjust the separation and pretreatment of the gaseous stream, while continuously obtaining the high quality propane composition.

After subjecting the hydrotreatment feed, especially comprising vegetable oils, animal fats and/or microbial oils, to the catalytic hydrotreatment, especially comprising at least hydrodeoxygenation as for example described above, propane will be present in the hydrotreatment effluent as one of a variety of gas phase components. The hydrotreatment effluent may then be subjected to gas-liquid separation to obtain at least a gaseous stream, comprising for example H₂, H₂S, CO, CO₂, H₂O, methane, ethane, propane and/or hydrocarbons having a carbon number of at least C4, and optionally a liquid hydrotreated stream, typically comprising C6-C30 hydrocarbons. Conveniently, the gaseous stream is subjected to a pretreatment, so as to separate e.g. a H₂ rich stream, thereby obtaining the gaseous composition.

In preferred embodiments, the gas-liquid separation is carried out at a temperature selected from a range from 0° C. to 500° C., preferably from 15° C. to 300° C., more preferably from 15° C. to 150° C., even more preferably from 15° C. to 65° C., and preferably at a pressure selected from a range from 1 to 200 bar (gauge), more preferably from 10 to 100 bar (gauge), or from 30 to 70 bar (gauge). The higher the pressure and/or the lower the temperature in the gas-liquid separation step, the lower the amount of heavy components (e.g. C4+ hydrocarbons) in the gaseous stream and in the gaseous composition.

The gas-liquid separation may be carried out as a separate step after the hydrotreatment effluent has left the hydrotreatment reactor or reaction zone and/or as an integral step e.g. within the hydrotreatment reactor or reaction zone. Majority of the water formed during HDO and potentially carried-over from the fresh hydrotreatment feed may be removed for example via a water boot in the gas-liquid separation step, while typically traces entrain in the gaseous stream.

The gaseous stream obtained by subjecting a hydrotreatment effluent to gas-liquid separation may contain hydrotreatment reaction products, such as H₂O, CO₂ and CO from the HDO and/or decarb-reactions, H₂ that has not been consumed in the hydrotreatment, H₂S generated from additives for catalyst sulphidation or sulphur-containing compounds in the hydrotreatment feed, NH₃ generated from nitrogen-containing compounds in the hydrotreatment feed, methane, ethane, propane and C4+ hydrocarbons generated by cracking of the hydrotreatment feed and the optional diluent, and propane generated from HDO of the glyceridic moiety present in hydrotreatment feeds comprising fatty acid glycerides. The content of propane in the gaseous stream may be enhanced for example by using hydrotreatment feed mainly containing fatty acid glycerides, by reducing the amount of the optional diluent in the hydrotreatment feed, by increasing the severity of the hydrotreatment conditions, and/or by using a catalyst or co-catalyst having higher cracking activity and/or selectivity.

The gaseous stream or the gaseous composition comprises entrained hydrocarbons having a carbon number of at least C4. In certain situations, for example if the hydrotreatment comprises hydrodeoxygenation and hydroisomerisation under conditions optimized for producing an aviation fuel component, or if the hydrotreatment comprises hydrodeoxygenation utilizing a catalyst or co-catalyst having elevated cracking tendency, the gaseous stream or gaseous composition may comprise a relatively high amount of C4+ hydrocarbons.

The C4+ hydrocarbons entrained in the gaseous stream or gaseous composition may comprise C4 to C6 hydrocarbons, including, but not limited to: butane, 2-methylpropane, pentane, isopentane, neopentane, hexane, 2-methylpentane, 3-methylpentane, 2,3-dimethylbutane, 2,2-dimethylbutane. In addition, the gaseous stream or gaseous composition may comprise hydrocarbons having seven or more carbon atoms, for example C7-C10 hydrocarbons, but their amount is typically very low.

The gaseous stream contains preferably at least 1 mol-% propane, such as at least 3 mol-% propane and/or 25 mol-% or less propane, such as 20 mol-% or less, or 15 mol-% or less, based on the total amount of substance of the gaseous stream. In embodiments, wherein the gaseous stream is derived from a hydrotreatment feed comprising vegetable oils, animal fats and/or microbial oils and having a glycerol-equivalent content of 2 wt-% to 60 wt-% relative to the total weight of the hydrotreatment feed, the propane content of the gaseous stream is often 25 mol-% or less. The term “glycerol-equivalent content relative to the total weight of the hydrotreatment feed” means a content of glycerol and/or glycerol-based moieties in the hydrotreatment feed and is calculated as if all glycerol (or glycerol-based) moieties (i.e. glycerol moieties in free glycerol and/or in monoglycerides, diglycerides or triglycerides, and/or glycerol-based moieties of e.g. partially deoxygenated glycerol, such as 1-propanol, 2-propanol, 1,2-propane diol or 1,3-propane diol and/or esters of these) were present as deprotonated glycerol (M=89.07 g/mol). In other words, the glycerol-equivalent content may be calculated as follows: glycerol-equivalent content=(Molar amount of glycerol-based moieties [mol])*89.07 g/mol/(total mass of the hydrotreatment feed [g]).

The liquid hydrotreated stream obtainable from the gas-liquid separation may comprise at least C6-C30 hydrocarbons, typically mainly C6-C30 paraffins. Preferably the liquid hydrotreated stream comprising C6-C30 hydrocarbons is subjected to a further catalytic hydrotreatment comprising at least hydroisomerisation. In these embodiments liquid fuel components having excellent cold properties are obtainable. A further gaseous stream separated from the further hydrotreatment effluent from the further hydrotreatment may optionally be combined with the gaseous stream from the main (first) hydrotreatment, before or after subjecting to pretreatment.

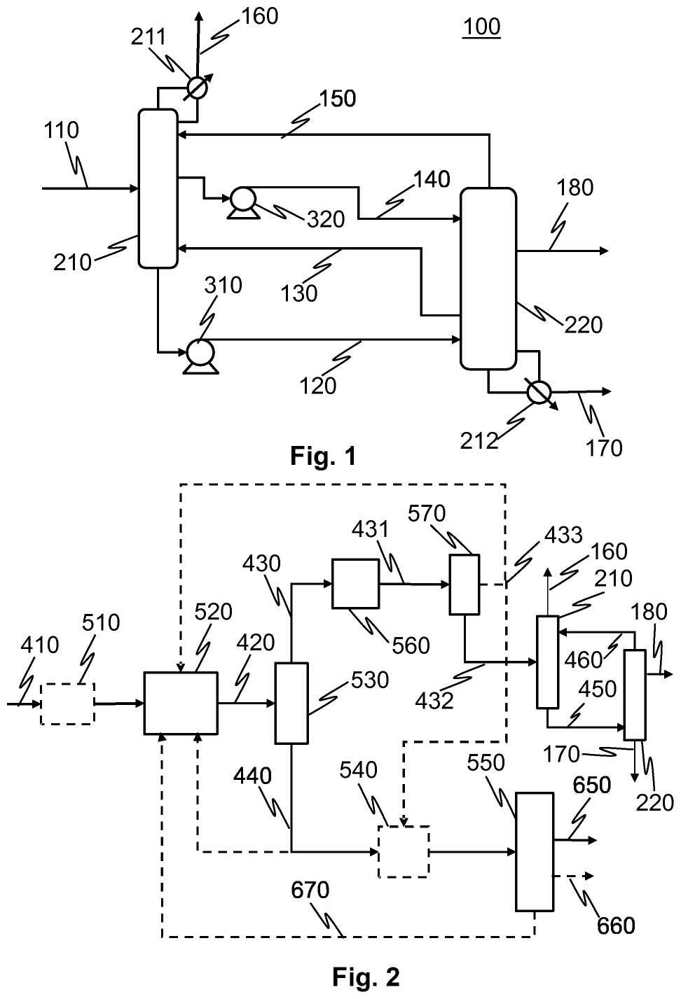

FIG. 1 shows a schematic drawing of distillation of step (ii) according to an example embodiment. In FIG. 1, a gaseous composition 110 such as a dried H₂S, CO₂, and H₂ depleted gaseous stream (432 in FIG. 2) as described in the foregoing is fed to a thermally coupled distillation system 100. The gaseous composition 110 is fed to a first distillation column 210 provided with a condenser 211 but without a reboiler (condenser column) configured to separate compounds lighter than propane. Below the inlet of the gaseous composition, the condenser column 210 is referred to as the bottom section of the condenser column, and above said feed inlet, the condenser column 210 is referred to as the top section of the condenser column. Condenser column bottom is pumped from the condenser column 210 with a first pump 310 as a liquid feed 120 to a bottom section of a second distillation column 220 provided with a reboiler 212 but without a condenser (reboiler column) and configured to separate compounds heavier than propane. Below a product outlet (propane composition outlet), the reboiler column 220 is referred to as the bottom section of the reboiler column, and above the product outlet, the reboiler column 220 is referred to as the top section of the reboiler column. From the bottom section of the reboiler column 220, a vapour boilup 130 is conducted to the bottom section of the condenser column 210, preferably using pressure difference between the reboiler and condenser columns 220, 210 as the driving force. Reboiler column overhead is conducted as a vapour feed 150 from the reboiler column 220 to the top section of the condenser column 210, preferably using pressure difference between the reboiler and condenser columns 220, 210 as the driving force. From the top section of the condenser column, below the inlet of the vapour feed 150, a liquid reflux 140 is pumped with a second pump 320 to the top section of the reboiler column 220. A stream of light compounds comprising e.g. H₂, CO, CO₂, CH₄, and ethane, exits as the condenser column overhead vapour 160, and a stream of heavy compounds comprising C4+ hydrocarbons exit as reboiler column bottom 170, from the thermally coupled distillation system 100. A propane composition 180 is recovered as a side draw from the product tray of the reboiler column 220.

FIG. 2 shows a schematic drawing of an example embodiment of the method of the present disclosure. In FIG. 2 some of the optional steps or treatments are denoted by dashed lines. In FIG. 2, there is provided a hydrotreatment feed 410 of vegetable oils, animal fats and/or microbial oils as described in the foregoing in connection with step (i), which hydrotreatment feed 410 is optionally subjected to purifying pretreatment 510. The optionally purified hydrotreatment feed 410 is fed to hydrotreatment 520. The hydrotreatment effluent 420 is fed to gas-liquid separation 530 to obtain a gaseous stream 430 and a liquid hydrotreated stream 440. At least a portion of the liquid hydrotreated stream 440 is subjected to fractionation 550, optionally after having been subjected to a further hydrotreatment 540 as described in the foregoing, to recover from the fractionation 550 at least an aviation fuel component 650, and optionally also a diesel fuel component 660 and/or a gasoline fuel component (not shown in FIG. 2). The effluent from the further hydrotreatment step, namely from hydrotreatment of the liquid hydrotreated stream 440, may be fed to a gas-liquid separation (not shown in FIG. 2) to obtain a further gaseous stream and a further liquid hydrotreated stream. A portion of the further liquid hydrotreated stream (not shown in FIG. 2) without having been subjected to the fractionation 550 and/or a further fraction 670 of hydrocarbons from the fractionation 550 may optionally be recycled to the hydrotreatment 520 as diluent. Optionally, a portion of the liquid hydrotreated stream 440 may be recycled to the hydrotreatment 520 without having been subjected to the further hydrotreatment 540 or the fractionation 550. The gaseous stream 430 is fed to a pretreatment comprising purification 560 to remove at least H₂S and CO₂ from the gaseous stream 430 thus obtaining a H₂S and CO₂ depleted gaseous stream 431. The H₂S and CO₂ depleted gaseous stream 431 is fed as part of the pretreatment to H₂ separation and drying 570 to obtain a dried H₂S, CO₂ and H₂ depleted gaseous stream 432 and a stream rich in H₂ 433. The stream rich in H₂ 433 (or a portion thereof) is optionally recycled to the hydrotreatment 520 and/or further hydrotreatment 540. The dried H₂S, CO₂ and H₂ depleted gaseous stream 432 is fed as the gaseous composition to fractional distillation in a thermally coupled distillation system comprising a condenser column 210 and a reboiler column 220 to recover a propane composition 180, in which thermally coupled distillation system the condenser column 210 is configured to provide liquid stream(s) 450 (liquid feed 120 and liquid reflux 140) to the reboiler column 220 and the reboiler column 220 is configured to provide vapour stream(s) 460 (vapour boilup 130 and reboiler column overhead (vapour feed) 150) to the condenser column 210. Condenser column 210 overhead vapour 160, i.e. stream of light compounds comprising e.g. H₂, CO, CO₂, CH₄, and ethane, and reboiler column 220 bottom 170, i.e. stream of heavy compounds comprising C4+ hydrocarbons, exit the thermally coupled distillation system.

### EXAMPLES

The following examples are provided to better illustrate the claimed invention and are not to be interpreted as limiting the scope of the invention. To the extent that specific materials are mentioned, it is merely for purposes of illustration and is not intended to limit the invention.

Separation of renewable, high purity propane compositions from gas compositions comprising different amounts of heavy tail, i.e. hydrocarbons having a carbon number of at least C4 (C4+ hydrocarbons), was studied.

A hydrotreatment feed comprising vegetable oils and animal fats, and a hydrocarbon diluent, was subjected to hydrodeoxygenation, followed by subjecting the hydrotreatment effluent to gas-liquid separation and aqueous phase removal to obtain a gaseous stream and a liquid hydrotreated stream. The gaseous stream comprised H₂, methane, ethane, propane, H₂O, H₂S, CO₂, CO, NH₃, and C4+ hydrocarbons, and was purified by subjecting to sweetening and ammonia removal, H₂ recovery and drying operations using the respective process steps as disclosed in WO2021110524, to provide a gaseous composition having main constituents as shown in Table 1. The biogenic carbon content of the provided gaseous composition was about 100 wt-% based on the total weight of carbon (TC) in the gaseous composition as determined according to EN 16640 (2017).

TABLE 1
Feed rate of gaseous composition to distillation and
main constituents thereof. C3 denotes propane.

| Feed to | | |
|---|---|---|
| distillation | kg/h | 9517 |
| H₂ | wt-% | 5.6 |
| N₂ | wt-% | <1 |
| CO | wt-% | 5.8 |
| CO₂ | wt-% | <1 |
| CH₄ | wt-% | 2.9 |
| C2 | wt-% | 2.0 |
| C3 | wt-% | 73.2 |
| C4 | wt-% | 4.5 |
| C5 | wt-% | 2.9 |
| C6+ | wt-% | 2.2 |

Simulation was performed with ASPEN PLUS on the gaseous composition of Table 1, using 9517 kg/h as the feed rate to distillation. With this feed rate, the rate of heavy tail of C4+ hydrocarbons in the gaseous composition of Table 1 was approximately 910 kg/h. Maximum allowed C4+ hydrocarbon content in the produced propane composition was set to 0.3 wt-%, while targeting at propane contents of about 96 wt-%. Propane compositions with such a high propane content and low C4+ hydrocarbon content are desired for example for use in catalytic upgrading.

In the simulation, the above-mentioned gaseous composition as the distillation feed was subjected to distillation in a heat integrated or thermally coupled distillation system corresponding to the distillation system shown in FIG. 1 having two columns: one condenser column provided with one condenser but not a reboiler, having <20 theoretical plates, and the combination of the condenser column (first distillation column) and the condenser operated using a temperature gradient of about 150° C. selected from −70° C. to 120° C. and a pressure gradient of about 50 kPa selected from 3200 to 3600 kPa; and one reboiler column (second distillation column) provided with one reboiler but not a condenser, having >20 theoretical plates, and the combination of the reboiler column and the reboiler operated using a temperature gradient of about 90° C. selected from 0° C. to 180° C. and a pressure gradient of <20 kPa selected from 3300 to 3700 kPa. The lowest pressure in the combination of the reboiler column and the reboiler was set to about 50 kPa higher than the highest pressure in the combination of the condenser column and condenser, and the reboiler duty was set to an initial value of −702 kW. The vapour flow between the columns was unidirectional from the reboiler column to the condenser column, and the liquid flow between the columns was unidirectional from the condenser column to the reboiler column.

Further simulations were performed by increasing the amount of heavy tail (C4+) gradually, illustrating situations where e.g. the hydrotreatment conditions, catalyst and/or feed composition are such that promote cracking of the feed, thereby increasing the amount of heavy tail in the gaseous stream separated from the hydrotreatment effluent. Chemical compositions and yields of the recovered renewable propane compositions, as well as propane recoveries, are shown in Table 2.

TABLE 2
Propane compositions recovered from distillation by a thermally
coupled distillation system with the initial reboiler duty, and
corresponding propane composition yields. C3 denotes propane.

| | | Thermally coupled distillation | | | | | | % of yield |
|---|---|---|---|---|---|---|---|---|
| Rate of | Amount of | | | | | Yield of | | with no |
| additional | Heavy tail | CO₂ | | | | the propane | C3 | additional |
| Heavy | in the feed | wt- | C3 | C4+ | C5+ | composition | recovery | heavy tail* |
| tail kg/h | wt-% | ppm | wt-% | wt-% | wt-% | kg/h | wt-% | % |
| 0 | 9.6 | 444 | 96.20 | 3.00 | 0.07 | 6935 | 95.7 | |
| 9 | 9.7 | 445 | 96.20 | 3.00 | 0.07 | 6933 | 95.7 | 100% |
| 46 | 10.0 | 449 | 96.20 | 3.00 | 0.07 | 6923 | 95.5 | 100% |
| 91 | 10.5 | 455 | 96.20 | 3.00 | 0.07 | 6911 | 95.4 | 100% |
| 137 | 11.0 | 461 | 96.18 | 3.00 | 0.07 | 6896 | 95.2 | 99% |
| 182 | 11.5 | 467 | 96.17 | 3.00 | 0.06 | 6880 | 94.9 | 99% |
| 273 | 12.4 | 481 | 96.15 | 3.00 | 0.06 | 6837 | 94.3 | 99% |
| 364 | 13.4 | 495 | 96.13 | 3.00 | 0.05 | 6773 | 93.4 | 98% |
| 455 | 14.3 | 511 | 96.11 | 3.00 | 0.04 | 6673 | 92.0 | 96% |
| 546 | 15.3 | 528 | 96.08 | 3.00 | 0.04 | 6512 | 89.8 | 94% |
| 637 | 16.3 | 550 | 96.04 | 3.00 | 0.03 | 6264 | 86.3 | 90% |
| 728 | 17.2 | 579 | 95.98 | 3.00 | 0.03 | 5881 | 81.0 | 85% |
| 819 | 18.2 | 628 | 95.87 | 3.00 | 0.03 | 5194 | 71.4 | 75% |
| 828 | 18.3 | 636 | 95.85 | 3.00 | 0.03 | 5086 | 69.9 | 73% |
| 837 | 18.4 | 645 | 95.83 | 3.00 | 0.03 | 4965 | 68.3 | 72% |
| 846 | 18.5 | 655 | 95.81 | 3.00 | 0.03 | 4825 | 66.3 | 70% |
| 856 | 18.6 | 667 | 95.78 | 3.00 | 0.03 | 4660 | 64.0 | 67% |
| 865 | 18.7 | 682 | 95.74 | 3.00 | 0.03 | 4459 | 61.2 | 64% |
| 874 | 18.7 | 703 | 95.69 | 3.00 | 0.03 | 4200 | 57.7 | 61% |
| 883 | 18.8 | 734 | 95.61 | 3.00 | 0.03 | 3828 | 52.5 | 55% |

*[Yield at x kg/h of additional heavy tail]/[Yield at 0 kg/h of additional heavy tail]

As seen in Table 2, the thermally coupled distillation system can be operated to obtain renewable propane compositions having a high degree of purity, and high propane content of more than 95 wt-%/around 96 wt-%, based on the total weight of the renewable propane composition, regardless of the amount of heavy tail in the distillation feed. Also, the amounts of C4+ hydrocarbons remaining in the obtained propane compositions were able to be kept low/in the target. Particularly, it was possible to keep the amounts of hydrocarbons having a carbon number of at least C5 (C5+ hydrocarbons) low. The yields of the propane compositions were also excellent, above 6.0 t/h (corresponding to about 90% or more of the yield recoverable with no additional heavy tail), when the added additional heavy tail was up to about 700 kg/h (corresponding to 04+ hydrocarbon content in the feed of about 17.0 wt-%). When the amount of additional heavy tail was about 850 kg/h or more (corresponding to 04+ hydrocarbon content in the feed of more than 18.5 wt-%), the yield of the propane composition dropped to less than 70% of the yield recoverable with no additional heavy tail. In case the feed would contain more additional heavy tail than 883 kg/h (corresponding to 04+ hydrocarbon content in the feed of more than 18.8 wt-%), the reboiler duty would need to be increased, making the process less energy-efficient. To demonstrate this, additional series of simulations were performed with 28.2% higher reboiler duty (results shown in Table 3).

TABLE 3
Propane compositions recovered from distillation by a thermally
coupled distillation system with 28.2% higher reboiler duty, and
corresponding propane composition yields. C3 denotes propane.

| | | Thermally coupled distillation | | | | | | % of yield |
|---|---|---|---|---|---|---|---|---|
| Rate of | Amount of | | | | | Yield of | | with no |
| additional | Heavy tail | CO₂ | | | | the propane | C3 | additional |
| Heavy | in the feed | wt- | C3 | C4+ | C5+ | composition | recovery | heavy tail* |
| tail kg/h | wt-% | ppm | wt-% | wt-% | wt-% | kg/h | wt-% | % |
| 819 | 18.2 | 463 | 96.18 | 3.00 | 0.13 | 6332 | 87.4 | 91% |
| 828 | 18.3 | 464 | 96.18 | 3.00 | 0.13 | 6327 | 87.3 | 91% |
| 837 | 18.4 | 464 | 96.18 | 3.00 | 0.13 | 6322 | 87.2 | 91% |
| 846 | 18.5 | 465 | 96.18 | 3.00 | 0.12 | 6317 | 87.2 | 91% |
| 856 | 18.6 | 466 | 96.18 | 3.00 | 0.12 | 6312 | 87.1 | 91% |
| 865 | 18.7 | 466 | 96.18 | 3.00 | 0.12 | 6307 | 87.0 | 91% |
| 874 | 18.7 | 467 | 96.17 | 3.00 | 0.12 | 6302 | 87.0 | 91% |
| 883 | 18.8 | 468 | 96.17 | 3.00 | 0.12 | 6297 | 86.9 | 91% |
| 892 | 18.9 | 468 | 96.17 | 3.00 | 0.12 | 6293 | 86.8 | 91% |
| 901 | 19.0 | 469 | 96.17 | 3.00 | 0.12 | 6288 | 86.8 | 91% |
| 910 | 19.1 | 470 | 96.17 | 3.00 | 0.12 | 6283 | 86.7 | 91% |
| 1001 | 20.1 | 477 | 96.16 | 3.00 | 0.11 | 6237 | 86.0 | 90% |
| 1092 | 21.0 | 484 | 96.14 | 3.00 | 0.10 | 6192 | 85.4 | 89% |
| 1183 | 22.0 | 491 | 96.13 | 3.00 | 0.09 | 6145 | 84.7 | 89% |
| 1274 | 22.9 | 500 | 96.11 | 3.00 | 0.08 | 6093 | 84.0 | 88% |
| 1365 | 23.9 | 509 | 96.10 | 3.00 | 0.07 | 6030 | 83.1 | 87% |

*[Yield at x kg/h of additional heavy tail]/[Yield at 0 kg/h of additional heavy tail]

From Table 3 it can be seen that with the higher energy consumption (reboiler duty) the targeted high propane content and low C4+ hydrocarbon content in the propane composition were reached regardless of the amount of additional heavy tail in the gaseous composition, even when the C4+ hydrocarbon content in the gaseous composition was as high as about 25 w %. However, the highest propane composition yields were at most 91% of the yield recoverable with no additional heavy tail added. There is thus an optimum in the heavy tail content (max 16.0 wt-%) in the gaseous composition (distillation feed) above which propane composition yields decrease regardless of increased reboiler duty.

As comparative simulation tests, four of the renewable distillation feeds with lowest heavy tail contents as described in Table 2 above were fed, respectively, to a elevated pressure distillation system as disclosed in WO2017045791 or WO2021110524 having one cryogenic distillation column provided with both a condenser and a reboiler. The renewable propane compositions recovered from the comparative distillation of each distillation feed and yields thereof are shown in Table 4.

TABLE 4
Comparative propane compositions recovered from comparative distillation
and corresponding propane yields. C3 denotes propane.

| | | | | | | | | % of |
|---|---|---|---|---|---|---|---|---|
| | | | | | | | | inventionex |
| | | Comparative distillation | | | | | | ample yield |
| Rate of | Amount of | | | | | Yield of | | with no |
| additional | Heavy tail | CO₂ | | | | the propane | C3 | additional |
| Heavy | in the feed | wt- | C3 | C4+ | C5+ | composition | recovery | heavy tail* |
| tail kg/h | wt-% | ppm | wt-% | wt-% | wt-% | kg/h | wt-% | % |
| 0 | 9.6 | 26 | 96.94 | 3.00 | 0.20 | 6838 | 95.1 | 99% |
| 9 | 9.7 | 26 | 96.94 | 3.00 | 0.20 | 6835 | 95.1 | 99% |
| 46 | 10.0 | 26 | 96.94 | 3.00 | 0.20 | 6825 | 94.9 | 98% |
| 91 | 10.5 | 26 | 96.94 | 3.00 | 0.20 | 6812 | 94.7 | 98% |

*[Yield at x kg/h of additional heavy tail]/[Yield at 0 kg/h of additional heavy tail (Table 2)]

From Table 4 it can be seen that while it was possible to reach the targeted C4+ hydrocarbon content in the propane composition, reaching it increased the propane content as well, which, when not necessary for the targeted use of the propane composition, leads to over-quality and decreases yields of the propane composition. The yields of the propane composition recoverable with the comparative distillation are slightly lower compared to the yield recoverable with the thermally coupled distillation system from a feed without additional heavy tail added, and the decrease in the yields along increased additional heavy tail in the feed is greater compared to the decrease in the yields recoverable by the thermally coupled distillation system. Also the propane recovery, when using the comparative distillation, is lower than when using the thermally coupled distillation system. Furthermore, C5+ hydrocarbon contents reached using the comparative distillation were on a higher level compared to Tables 2 and 3, which may render the propane compositions reported in Table 4 unsuitable for certain uses.

In addition to the improved propane composition yields, significant savings in condenser and reboiler duty were achieved with the thermally coupled distillation compared to the comparative distillation. The condenser and reboiler duties, respectively, of the distillations reported in Tables 4 and 2 are shown in Table 5.

TABLE 5
Condenser and reboiler duties of the thermally coupled distillation
system and the comparative distillation system.

| | Comparative | | Thermally coupled | | | |
|---|---|---|---|---|---|---|
| Heavy | distillation | | distillation | | Savings | Savings |
| tail | QC | QR | QC | QR | QC | QR |
| kg/h | kW | kW | kW | kW | kW | kW |
| 0 | 4469 | 4682 | 939 | 702 | 3530 | 3743 |
| 9 | 4708 | 4495 | 938 | 702 | 3770 | 3557 |
| 46 | 4811 | 4602 | 933 | 702 | 3878 | 3669 |
| 91 | 4942 | 4737 | 927 | 702 | 4015 | 3810 |

QC = condenser duty;
QR = reboiler duty

As seen in Table 5, the savings in condenser duty range from roughly 3500 kW to about 4000 kW, and in reboiler duty from roughly 3700 kW to about 3800 kW. The savings in condenser and in reboiler duty increase along with increased amount of heavy tail in the distillation feed.

Distillation with a thermally coupled distillation system comprising a condenser column and a reboiler column thus provides high quality, on-specification renewable propane compositions with improved propane composition yields, and significant savings in reboiler and condenser duties compared to obtaining high quality renewable propane compositions through comparative distillation with one cryogenic distillation column.

Various embodiments have been presented. It should be appreciated that in this document, words comprise, include, and contain are each used as open-ended expressions with no intended exclusivity.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1-21. (canceled)

22. A method for treating a gaseous composition, comprising:
(i) providing a gaseous composition including H₂, methane, ethane, propane, and hydrocarbons having a carbon number of at least C4, wherein a total amount of H₂, methane, ethane, propane, and hydrocarbons having a carbon number of at least C4 is at least 80 wt-%; and
(ii) subjecting the gaseous composition to distillation in a thermally coupled distillation system including n distillation columns and at least one and at most n−1 condenser(s) and at least one and at most n−1 reboiler(s), n being an integer ≥2, to recover a propane composition.

23. The method according to claim 22, wherein the thermally coupled distillation system includes:
at least a first distillation column connected to a condenser without being connected to a reboiler, and a second distillation column connected to a reboiler without being connected to a condenser, and the method comprising:
feeding the gaseous composition to the first distillation column; and
recovering the propane composition from the second distillation column.

24. The method according to claim 22, comprising:
providing a liquid feed and a liquid reflux from the first distillation column to the second distillation column of the thermally coupled distillation system; and
providing a vapour feed and a vapour boilup from the second distillation column to the first distillation column of the thermally coupled distillation system.

25. The method according to claim 24, comprising:
providing the first column bottom as the liquid feed to the bottom section of the second column;
providing the second column overhead as the vapour feed to the top section of the first column;
providing the liquid reflux from the top section of the first column, below the inlet of the vapour feed, to the top section of the second column; and
providing the vapour boilup from the bottom section of the second column, above the inlet of the liquid feed, to the bottom section of the first column.

26. The method according to claim 22, wherein the first distillation column of the thermally coupled distillation system is configured to separate compounds lighter than propane, and the second distillation column of the thermally coupled distillation system is configured to separate compounds heavier than propane.

27. The method according to claim 22, wherein the gaseous composition has a content ratio of hydrocarbons having a carbon number of at least C4 to propane of at least 0.05.

28. The method according to claim 22, wherein the gaseous composition comprises:
hydrocarbons having a carbon number of at least C5, and has a content ratio of hydrocarbons having a carbon number of at least C5 to propane of at least 0.01.

29. The method according to claim 22, wherein the gaseous composition comprises:
hydrocarbons having a carbon number of at least C4 at most 18.5 wt-%, and/or at least 5.0 wt-%, based on a total weight of the gaseous composition; and/or
hydrocarbons having a carbon number of at least C5 at most 15.0 wt-%, and/or at least 1.5 wt-%, based on the total weight of the gaseous composition.

30. The method according to claim 22, wherein the gaseous composition comprises:
propane at least 60 wt-%, based on a total weight of the gaseous composition.

31. The method according to claim 22, wherein the gaseous composition comprises:
compounds lighter than propane at most 35 wt-%, based on a total weight of the gaseous composition, and/or H₂ from 1 to 10 wt-%, based on the total weight of the gaseous composition.

32. The method according to claim 22, wherein the propane composition comprises:
based on a total weight of the propane composition, at least 95 wt-% propane, and at most 5.0 wt-% hydrocarbons having a carbon number of at least C4, and at most 0.20 wt-% hydrocarbons having a carbon number of at least C5.

33. The method according to claim 22, wherein the gaseous composition has a biogenic carbon content of at least 50 wt-%, based on a total weight of carbon (TC) in the gaseous composition; and/or
the propane composition has a biogenic carbon content of at least 50 wt-% based on the total weight of carbon (TC) in the propane composition.

34. The method according to claim 22, wherein the thermally coupled distillation system is operated at pressures above 1500 kPa, and/or at temperatures above −70° C.

35. The method according to claim 22, wherein a combination of the first distillation column of the n distillation columns and a condenser of the n−1 condensers is operated at pressures within a range from 1500 kPa to 5000 kPa, and at temperatures within a range from −70° C. to 120° C.

36. The method according to claim 22, wherein:
a lowest pressure in a combination of a second distillation column of the n distillation columns and a reboiler of the n−1 reboilers is higher than a highest pressure in a combination of a first distillation column and a condenser of the n−1 condensers.

37. The method according to claim 22, wherein:
a lowest temperature in a combination of a first distillation column of the n distillation columns and a condenser of the n−1 condensers is below 0° C., and a highest temperature in a combination of a second distillation column and a reboiler of the n−1 reboilers is higher than a highest temperature in the combination of the first distillation column and the condenser.

38. The method according to claim 22, wherein (i) providing the gaseous composition comprises:
subjecting a hydrotreatment effluent to gas-liquid separation to obtain at least a gaseous stream, and subjecting the gaseous stream to a pretreatment to obtain the gaseous composition.

39. The method according to claim 22, wherein (i) providing the gaseous composition comprises:
subjecting a hydrotreatment feed including vegetable oils, animal fats, microbial oils, crude oil, thermally and/or enzymatically liquefied organic waste and residues, or a combination thereof, to a catalytic hydrotreatment including hydrodeoxygenation, hydrocracking, hydroisomerization, hydropyrolysis, hydrodesulphurization, hydrodenitrogenation, hydrodehalogenisation, hydrodearomatization, hydrodemetallation and/or hydrogenation, to obtain a hydrotreatment effluent;
subjecting the hydrotreatment effluent to gas-liquid separation to obtain at least a gaseous stream; and
subjecting the gaseous stream to a pretreatment to obtain the gaseous composition.

40. The method according to claim 22, wherein (i) providing the gaseous composition comprises:
subjecting a hydrotreatment feed including vegetable oils, animal fats and/or microbial oils to a catalytic hydrotreatment including at least hydrodeoxygenation using a sulphided HDO catalyst, to obtain a hydrotreatment effluent;
subjecting the hydrotreatment effluent to gas-liquid separation to obtain at least a gaseous stream including H₂, H₂S, CO, CO₂, H₂O, methane, ethane, propane and hydrocarbons having a carbon number of at least C4, and a liquid hydrotreated stream including C6-C30 hydrocarbons; and
subjecting the gaseous stream to a pretreatment including at least purification to remove H₂S and CO₂, H₂ separation and drying, to obtain dried H₂S, CO₂ and H₂ depleted gaseous stream as the gaseous composition; and
wherein the method includes subjecting the liquid hydrotreated stream including C6-C30 hydrocarbons, or the liquid hydrotreated stream including C6-C30 hydrocarbons which has been subjected to a further catalytic hydrotreatment including at least hydroisomerisation, to a fractionation to recover one or more of a gasoline fuel component, an aviation fuel component, and/or a diesel fuel component.

41. A method for co-producing a propane composition and a fuel component comprising:
(i) subjecting a hydrotreatment feed including vegetable oils, animal fats and/or microbial oils to a catalytic hydrotreatment including at least hydrodeoxygenation using a sulphided hydrotreatment catalyst, to obtain a hydrotreatment effluent;
subjecting the hydrotreatment effluent to gas-liquid separation to obtain at least a gaseous stream including H₂, H₂S, CO, CO₂, H₂O, methane, ethane, propane and hydrocarbons having a carbon number of at least C4, and a liquid hydrotreated stream including C6-C30 hydrocarbons;
subjecting the gaseous stream to a pretreatment including at least purification to remove H₂S and CO₂, H₂ separation and drying to obtain a gaseous composition including H₂, methane, ethane, propane, and hydrocarbons having a carbon number of at least C4, wherein a total amount of H₂, methane, ethane, propane, and hydrocarbons having a carbon number of at least C4 is at least 80 wt-% of a total weight of the gaseous composition; and
(ii) subjecting the gaseous composition to distillation in a thermally coupled distillation system including at least a first distillation column connected to a condenser without being connected to a reboiler, and a second distillation column connected to a reboiler without being connected to a condenser, by feeding the gaseous composition to the first distillation column, and recovering a propane composition from the second distillation column; and
wherein the method includes subjecting the liquid hydrotreated stream including C6-C30 hydrocarbons, or the liquid hydrotreated stream including C6-C30 hydrocarbons which has been subjected to a further catalytic hydrotreatment including at least hydroisomerisation, to a fractionation to recover one or more of a gasoline fuel component, an aviation fuel component, and/or a diesel fuel component.
